(19) United States
(12) Patent Application Publication
(10) Pub. No.: US 2008/0167682 A1
(43) Pub. Date: Jul. 10, 2008

(54) Bioabsorbable occlusion device

(75) Inventors: Michael P. Corcoran, Woodbury, MN (US); Joseph A. Marino, Apple Valley, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Cardia, Inc., Eagan, MN (US)

(21) Appl. No.: 11/651,230

(22) Filed: Jan. 9, 2007

(51) Int. Cl. A61B 17/03

(52) U.S. Cl. 606/215; 606/213

(57) ABSTRACT

An occlusion device comprises a bioabsorbable material, which allows the occlusion device to effectively close a defect in the heart while eliminating or limiting components which permanently remain in the body.

### BACKGROUND OF THE INVENTION

This invention relates to an occlusion device for repairing cardiovascular defects. More specifically, this invention relates to an occlusion device comprising a bioabsorbable material, which allows the occlusion device to effectively close a defect in the heart while eliminating or limiting components which permanently remain in the body.

Normally, permanently repairing certain cardiac defects in adults and children requires open heart surgery, a risky, expensive, and painful procedure. To avoid the risks and discomfort associated with open heart surgery, occlusion devices have been developed that are small, implantable devices capable of being delivered to the heart through a catheter. Rather than surgery, a catheter inserted into a major blood vessel allows an occlusion device to be deployed by moving the device through the catheter. This procedure is performed in a cardiac cathlab and avoids the risks and pain associated with open heart surgery. These occlusion devices can repair a wide range of cardiac defects, including patent foramen ovale, patent ductus arteriosus, atrial septal defects, ventricular septal defects, and may occlude other cardiac and non-cardiac apertures. There are currently several types of occlusion devices capable of being inserted via a catheter.

Although tissue growth occurs over the defect site, once implanted, these occlusion devices remain in position throughout the life of the patient. Therefore, there is some concern that the components of the occlusion device which are under stress may break. Broken components increase the likelihood of damage to the surrounding tissue. In addition, since current occlusion devices are often formed of metallic components there is a possibility that corrosion will occur over a period of time.

Thus, there is a need in the art for an occlusion device that will occlude cardiac defects for a period during which tissue growth occurs and is then capable of being absorbed into the body after a sufficient amount of time.

### BRIEF SUMMARY OF THE INVENTION

The present invention allows effective closure of a cardiac defect, while eliminating or minimizing components which remain in the body permanently. The present invention is an occlusion device having first and second support frames comprising a plurality of arms, a center post extending between the first and second support frames, and first and second sheets attached to the first and second support frames, respectively. The components of the occlusion device are comprised of a bioabsorbable material.

### BRIEF DESCRIPTION OF THE DRAWINGS

FIGS. 1A and 1B illustrate a first embodiment of a bioabsorbable occlusion device in which the entire occlusion device is formed of a bioabsorbable material.

FIG. 2 is a second embodiment of a bioabsorbable occlusion device in which the sheets of the occlusion device are formed of a bioabsorbable material.

FIG. 3 is a third embodiment of a bioabsorbable occlusion device in which the sheets and center post of the occlusion device are formed of a bioabsorbable material.

### DETAILED DESCRIPTION

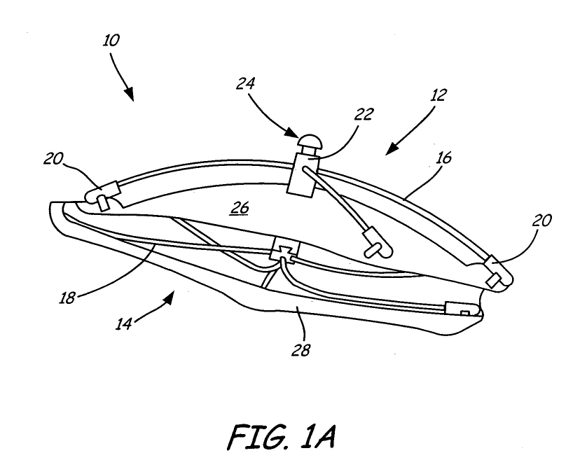

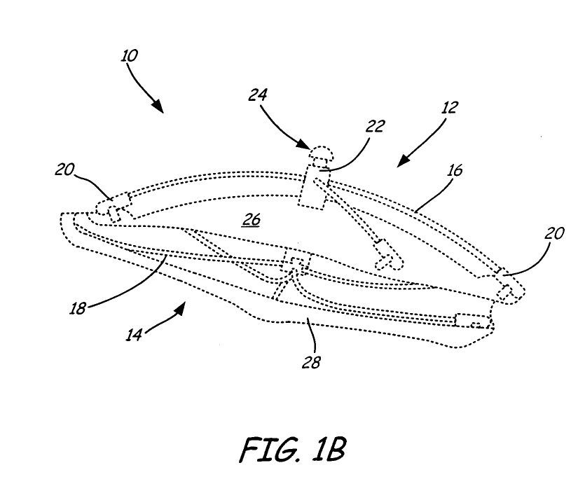

FIGS. 1A and 1B show an exemplary embodiment of occlusion device 10, which is entirely formed of bioabsorbable materials. FIG. 1A shows occlusion device 10 in solid lines to illustrate its condition at implantation. FIG. 1B shows occlusion device 10 in dashed lines, representing that the components of occlusion device 10 are absorbed over time. Occlusion device 10 includes proximal support frame 12, distal support frame 14, proximal arms 16, distal arms 18, atraumatic tips 20, center post 22, knob 24, proximal sheet 26, and distal sheet 28.

Proximal and distal support frames 12, 14 are comprised of arms 16, 18, respectively, and are attached to proximal and distal sheets 26, 28. Center post 22 extends between proximal and distal support frames 12, 14. Although in the exemplary embodiment shown in FIG. 1, proximal and distal support frames 12, 14 each comprise five arms 16, 18, proximal and distal support frames 12, 14 may comprise more or less arms 16, 18.

One method of connecting proximal and distal support frames 12, 14 to center post 22 is to provide center post 22 with holes through which arms 16, 18 extend. In the exemplary embodiment shown in FIG. 1, atraumatic tips 20 are located at the distal end of each arm 16, 18 and serve to minimize damage to the surrounding tissue. Atraumatic tips 20 may also be used to secure proximal and distal sheets 26, 28 to proximal and distal support frames 12, 14. One method of attaching proximal and distal sheets 26, 28 to proximal and distal support frames 12, 14 is to provide atraumatic tips 20 with holes through which sutures pass. In this way, proximal and distal sheets 26, 28 are sewn to proximal and distal support frames 12, 14 at atraumatic tips 20. Another method of securing proximal and distal sheets 26, 28 to proximal and distal support frames 12, 14 is to form proximal and distal sheets 26, 28 directly around proximal and distal arms 16, 18.

Center post 22 is preferably formed to have a diameter of less than about 10 millimeters and a length of preferably less than about 20 millimeters. The size of proximal and distal sheets 26, 28 may vary to accommodate various defect sizes. When measured diagonally, the size of proximal and distal sheets 26, 28 may range from about 15 millimeters to about 45 millimeters. In some instances, it may be desirable to form sheets 26, 28 so that they are not both the same size. For instance, one sheet and its associated support frame can be made smaller (25 millimeters, for example) than the opposing sheet and its associated fixation device (30 millimeters, for example). This is particularly useful in situations where occlusion device 10 is to be placed at a location in the heart, which is close to other nearby cardiac structures. Making sheets 26, 28 different sizes may assist in providing optimal occlusion of a defect, without affecting other structures of the heart, which may be nearby.

Occlusion device 10 is constructed so that proximal and distal support frames 12, 14 are easily collapsible about center post 22. Due to this construction, occlusion device 10 can be folded so that proximal and distal support frames 12, 14 are folded in an axial direction. Proximal and distal sheets 26, 28, which are attached to proximal and distal support frames 12, 14, are flexible, and can likewise collapse as proximal and distal support frames 12, 14 are folded. Once occlusion device 10 is deployed, proximal and distal support frames 12, 14 must serve to hold proximal and distal sheets 26, 28 in place to seal a defect. In addition, center post 22 further comprises knob 24, which allows for occlusion device 10 to be grasped by a delivery forceps as it is inserted into the body through a catheter.

To insert occlusion device 10, a catheter is positioned proximate the septal defect to be occluded. Next, a delivery forceps is used to push occlusion device 10 through the catheter so that distal sheet 26 attached to distal support frame 12 unfolds in the left atrium. Although the distal portion of occlusion device 10 has been deployed, proximal sheet 28 attached to proximal support frame 14 is still folded in the catheter. The proximal portion of occlusion device 10 unfolds as the catheter is withdrawn.

Another feature of occlusion device 10 is that it is fully retrievable. To allow occlusion device 10 to be retrievable, as well as ensure that occlusion device 10 fits into a small diameter catheter, it is preferable to ensure that the proximal and distal arms 16, 18 of proximal and distal support frames 12, 14 are not of a length that results in atraumatic tips 20 clustering at the same location. If atraumatic tips 20 all cluster at the same location when occlusion device 10 is inside a catheter, occlusion device 10 may be too bulky to allow it to be easily maneuvered through the catheter.

In the exemplary embodiment shown in FIG. 1, occlusion device 10 is formed entirely of a bioabsorbable material. Possible suitable bioabsorbable materials may include collagen or synthetic polymers, such as polylactides, polyglycolides, polycaprolactones, polycarbonates, polydioxanones, and polyactives. A combination or composite material may also be used to form occlusion device 10. Since collagen is a naturally occurring protein, it provides minimal antigenicity and readily allows for tissue attachment. Resorbable polymers also support cell growth and tissue generation. In addition, it is relatively easy to “dial in” specific resorption rates (the amount of time needed for the material to be absorbed by the body) for synthetic polymers. Depending upon the polymer family, resorption rates may be as short as a few weeks or as long as several years. Both collagen and synthetic polymers can be processed into a number of resorbable formations, including gels, pastes, powders, sponges, and coatings. A variety of resorbable materials are made commercially available by the Kensey Nash Corporation.

Proximal and distal support frames 12, 14, proximal and distal sheets 26, 28, and center post 22 may all be formed of the same bioabsorbable material or a combination thereof. For example, if it is desired that specific components of occlusion device 10 remain in the heart longer than others then occlusion device 10 may be formed of materials having varying resorption rates.

Center post 22 and proximal and distal support frames 12, 14 may be formed by any suitable method such as injection molding or machining. Proximal and distal sheets 26, 28 may be formed by a foaming method. It is also possible to mechanically process proximal and distal sheets 26, 28 by slicing a suitable bioabsorbable material in the desired shape. To minimize the chance of occlusion device 10 causing a blood clot, foam proximal and distal sheets 26, 28 may be treated with a thrombosis inhibiting material, such as heparin.

Forming occlusion device 10 entirely of bioabsorbable materials eliminates many of the concerns associated with conventional devices. For example, since the concern that that components of the occlusion device under pressure, such as proximal and distal arms 16, 18, may break increases over time, it is preferably to limit the amount of time these components remain in the heart. As a result, the likelihood of damage to the surrounding tissue is decreased. In addition, eliminating or limiting the use of metallic components nullifies or at least reduces the possibility that corrosion will occur over a period of time.

The bioabsorbable material used to form occlusion device 10 may also include a radiopaque component so it is possible for occlusion device 10 to be detected on an x-ray. In the alternative, occlusion device 10 may also be coated with a radiopaque material. It is important for occlusion device 10 to be visible so its position in the heart can be monitored during insertion.

In addition, the bioabsorbable material used to form occlusion device 10 may also be invested with drugs or other biologically active therapeutic agents. In that case, occlusion device 10 can be capable of controlled release of these drugs or agents for sustained or local delivery.

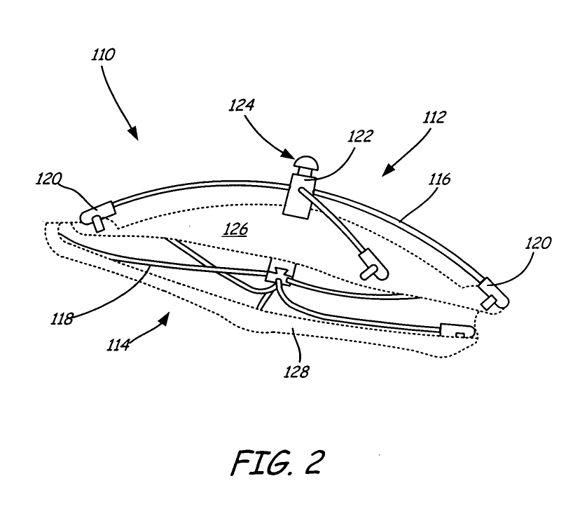

FIG. 2 is an exemplary embodiment of occlusion device 110, which is partially formed of a bioabsorbable material. The bioabsorbable components are shown in dashed lines. Occlusion device 110 includes proximal support frame 112, distal support frame 114, proximal arms 116, distal arms 118, atraumatic tips 120, center post 122, knob 124, proximal sheet 126, and distal sheet 128.

In the exemplary embodiment shown in FIG. 2, proximal and distal sheets 126, 128 are formed of a bioabsorbable material, such as collagen or a synthetic polymer. Proximal and distal support frames 112, 114 and center post 122 may be comprised of any suitable material, including Nitinol (a nickel-titanium alloy), titanium or stainless steel.

As described with reference to FIG. 1, proximal and distal sheets 126, 128 are attached to proximal and distal support frames 112, 114 and press against the walls of the heart to seal the defect. Since suitable bioabsorbable materials support cell growth, proximal and distal sheets 126, 128 are no longer needed to occlude the defect after adequate tissue generation has occurred. Also, since proximal and distal sheets 126, 128 make up a large amount of the exposed surface area of occlusion device 110, only a small portion of occlusion device 110 (specifically proximal and distal support frames 112, 114 and center post 122) remains after proximal and distal sheets 126, 128 are absorbed into the body. This elimination of a large percentage of the overall surface area of occlusion device 110 may minimize patient concerns about the amount of foreign material remaining in the heart for the remainder of his or her life.

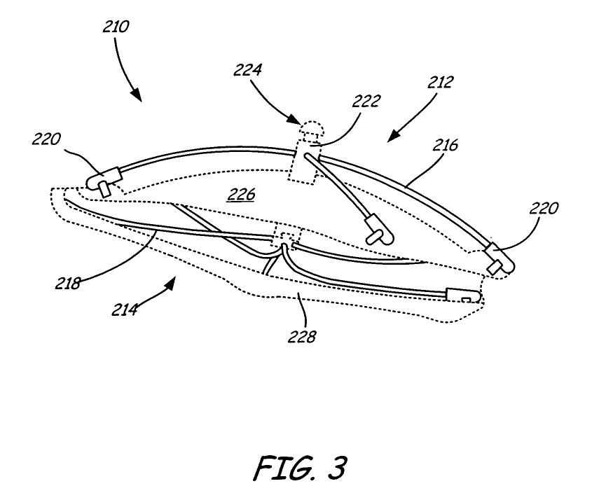

FIG. 3 is an exemplary embodiment of occlusion device 210, which is partially formed of a bioabsorbable material. The bioabsorbable components are shown in dashed lines. Occlusion device 210 includes proximal support frame 212, distal support frame 214, proximal arms 216, distal arms 218, atraumatic tips 220, center post 222, knob 224, proximal sheet 226, and distal sheet 228.

In the exemplary embodiment shown in FIG. 3, proximal and distal sheets 226, 228 and proximal and distal support frames 212, 214 are formed of a bioabsorbable material, such as collagen or a synthetic polymer. Center post 222 may be comprised of any suitable material, including Nitinol (a nickel-titanium alloy), titanium or stainless steel.

Often occlusion device 210 must occlude an irregularly shaped defect. For example, the septal wall on the bottom of septal defect may be only a few millimeters thick, but the septal wall on the top of septal defect may be much thicker. In such cases, one side of occlusion device 210 may be bent open further than the other side. The side that is more distorted carries a high static load, which increases pressure on the surrounding tissue and also increases the possibility of device fracture or septal tissue perforation. Forming proximal and distal arms 216, 218 of proximal and distal support frames 212, 214, in addition to proximal and distal sheets 226, 228, out of a bioabsorbable material decreases the likelihood of damage to tissue resulting from device fracture. Because occlusion device 210 remains in place for such a short amount of time, the fatigue life of occlusion device 210 is not an issue. Also, since proximal and distal support frames 212, 214 are formed of a bioabsorbable material, if device fracture does occur, broken fragments will be absorbed by the body over time.

Although the present invention has been described with reference to preferred embodiments, workers skilled in the art will recognize that changes may be made in form and detail without departing from the spirit and scope of the invention.

1. An occlusion device comprising:
first and second support frames comprising a plurality of arms;
a center post extending between the first and second support frames;
first and second sheets attached to the first and second support frames, respectively; and
wherein the occlusion device is comprised at least in part of a bioabsorbable material.

2. The occlusion device of claim 1 wherein the bioabsorbable material comprises a polymer.

3. The occlusion device of claim 2 wherein the polymer includes at least one of the following: a polylactide, a polyglycolide, a polycaprolactone, a polycarbonate, a polydioxanone, and a polyactive.

4. The occlusion device of claim 1 wherein the bioabsorbable material comprises collagen.

5. The occlusion device of claim 1 wherein the occlusion device further comprises a radiopaque material.

6. The occlusion device of claim 1 wherein the bioabsorbable material includes a deliverable biologically active agent.

7. An occlusion device comprising:
a center post;
first and second collapsible support frames connected to the center post; and
a first sheet formed of a bioabsorbable material attached to the first collapsible support frame.

8. The occlusion device of claim 7 and comprising a second sheet formed of a bioabsorbable material attached to the second collapsible support frame.

9. The occlusion device of claim 8 wherein the center post is formed of a bioabsorbable material.

10. The occlusion device of claim 7 wherein the center post is formed of a bioabsorbable material.

11. The occlusion device of claim 10 wherein the first and second collapsible support frames are formed of a bioabsorbable material.

12. The occlusion device of claim 11 wherein the first and second collapsible support frames include a radiopaque material.

13. The occlusion device of claim 7 wherein the bioabsorbable material comprises a polymer.

14. The occlusion device of claim 13 wherein the polymer includes at least one of the following: a polylactide, a polyglycolide, a polycaprolactone, a polycarbonate, a polydioxanone, and a polyactive.

15. The occlusion device of claim 7 wherein the bioabsorbable material comprises collagen.

16. The occlusion device of claim 7 wherein the bioabsorbable material includes a deliverable biologically active agent.

17. An occlusion device comprising:
a first fixation device configured to be placed on a first side of a septal defect, the first fixation device comprising a plurality of arms, each arm including an atraumatic tip;
a second fixation device configured to be placed on a second side of a septal defect, the second fixation device comprising a plurality of arms, each arm including an atraumatic tip; and
a center post connecting the first fixation device and the second fixation device;
first and second bioabsorbable sheets attached to the first and second fixation devices, respectively.

18. The occlusion device of claim 17 wherein the first and second bioabsorbable sheets are comprised of a polymer.

19. The occlusion device of claim 17 wherein the first and second bioabsorbable sheets are comprised of collagen.

20. The occlusion device of claim 17 wherein the center post is formed of a bioabsorbable material.

21. The occlusion device of claim 17 wherein the first and second fixation devices are formed of a bioabsorbable material.

22. The occlusion device of claim 17 wherein the center post and the first and second fixation devices are formed of a bioabsorbable material.
